(19) United States
(12) Patent Application Publication
(10) Pub. No.: US 2011/0009411 A1
(43) Pub. Date: Jan. 13, 2011

(54) THERAPEUTIC COMPOSITIONS AND THE USE THEREOF

(75) Inventors: Brian P. Kearney, Foster City, CA (US); Anita A. Mathias, Foster City, CA (US)

Correspondence Address: VIKSNINS HARRIS & PADYS PLLP, P.O BOX 111098, St. Paul, MN, 55111-1098, US

(73) Assignee: Gilead Sciences ,Inc., Foster City (CA)

(21) Appl. No.: 12/666,995

(22) PCT Filed: Jun. 26, 2008
(86) PCT No.: PCT/US2008/068351

§ 371 Date: Jul. 20, 2010

Related U.S. Application Data
(60) Provisional application No. 60/947,325, filed on Jun. 29, 2007.

(51) Int. Cl. A61K 31/47; A61K 31/513; A61K 31/5377; A61P 31/12; A61P 31/18

(52) U.S. Cl. 514/236.8; 514/312; 514/274

(57) ABSTRACT

The invention includes methods, compositions, and kits useful for treating a viral infection by coadministering 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof, with lopinavir or a pharmaceutically acceptable salt thereof.

### BACKGROUND OF THE INVENTION

A series of 4-oxoquinolines including the compound 6-(3-chloro-2-fluorobenzyl)-1-[(2S)-1-hydroxy-3-methylbutan-2-yl]-7-methoxy-4-oxo-1,4-dihydroquinoline-3-carboxylic acid (the Compound) have been identified as anti-human immunodeficiency virus (HIV) agents. See U.S. patent application Ser. No. 10/492,833, filed Nov. 20, 2003, which was published as United States Patent Application Publication Number 2005/0239819. Specifically, the Compound has been described as having inhibitory activity against the integrase protein of HIV. Id. HIV belongs to the retrovirus family and is a causative agent of the acquired immunodeficiency syndrome (AIDS). Accordingly, a pharmaceutical agent that reduces the virus load, viral genome, or replication of HIV in the body, may be effective for the treatment or prophylaxis of AIDS.

The treatment cost and the potential for unwanted side-effects can both increase as the required dose of a drug increases. Therefore, there is a need for methods and compositions that are useful for achieving an acceptable anti-viral effect using a reduced dose of the Compound.

### SUMMARY OF THE INVENTION

It has been determined that the systemic exposure to the Compound in humans improves when the Compound is administered with ritoavir-boosted lopinavir (LPV/r). A dose of 85±10 mg of the Compound administered with ritonavir-boosted lopinavir was calculated to have a systemic exposure equivalent to a 150 mg dose of the Compound alone.

Accordingly, in one embodiment the invention provides a method of treating a viral infection in a human comprising administering 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof, lopinavir, or a pharmaceutically acceptable salt thereof, and a compound that inhibits cytochrome P-450 (e.g. ritonavir) to the human.

In one embodiment the invention also provides a pharmaceutical composition comprising 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof; lopinavir or a pharmaceutically acceptable salt thereof; and a pharmaceutically acceptable carrier or diluent.

In one embodiment, the invention provides a use of the compound 6-(3-chloro-2-fluorobenzyl)-1-[(2S)-1-hydroxy-3-methylbutan-2-yl]-7-methoxy-4-oxo-1,4-dihydroquinoline-3-carboxylic acid or its pharmaceutically acceptable salt for the manufacture of a medicament for treating a viral infection in a human, comprising administering the compound or a pharmaceutically acceptable salt thereof, lopinavir, or a pharmaceutically acceptable salt thereof, and a compound that inhibits cytochrome P-450 (e.g. ritonavir) to the human.

In one embodiment, the invention provides the use of lopinavir, or a pharmaceutically acceptable salt thereof, to prepare a medicament useful for improving the pharmacokinetics of 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof, following administration to a human.

In one embodiment, the invention provides lopinavir for use in improving the pharmacokinetics of 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof, following administration to a human.

In one embodiment, the invention provides a kit comprising: (1) 6-(3-chloro-2-fluorobenzyl)-1-[(2S)-1-hydroxy-3-methylbutan-2-yl]-7-methoxy-4-oxo-1,4-dihydroquinoline-3-carboxylic acid, or a pharmaceutically acceptable salt thereof; (2) lopinavir, or a pharmaceutically acceptable salt thereof; (3) one or more containers; and (4) prescribing information regarding administering the 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof with the lopinavir or a pharmaceutically acceptable salt thereof.

In one embodiment, the invention provides a kit comprising: (1) a unit dosage form comprising 85±10 mg of 6-(3-chloro-2-fluorobenzyl)-1-[(2S)-1-hydroxy-3-methylbutan-2-yl]-7-methoxy-4-oxo-1,4-dihydroquinoline-3-carboxylic acid, or a pharmaceutically acceptable salt thereof; (2) lopinavir, or a pharmaceutically acceptable salt thereof; (3) one or more containers; and (4) prescribing information regarding administering the 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof with lopinavir or a pharmaceutically acceptable salt thereof.

In one embodiment, the invention provides a use of the compound 6-(3-chloro-2-fluorobenzyl)-1-[(2S)-1-hydroxy-3-methylbutan-2-yl]-7-methoxy-4-oxo-1,4-dihydroquinoline-3-carboxylic acid or its pharmaceutically acceptable salt for the manufacture of a medicament for inhibiting activity of a retroviral integrase in a human, comprising administering the compound or a pharmaceutically acceptable salt thereof, lopinavir, or a pharmaceutically acceptable salt thereof, and a compound that inhibits cytochrome P-450 (e.g. ritonavir) to the human.

In one embodiment, the invention provides 6-(3-chloro-2-fluorobenzyl)-1-[(2S)-1-hydroxy-3-methylbutan-2-yl]-7-methoxy-4-oxo-1,4-dihydroquinoline-3-carboxylic acid or its pharmaceutically acceptable salt; lopinavir, or a pharmaceutically acceptable salt thereof; and a compound that inhibits cytochrome P-450 for use in inhibiting activity of a retroviral integrase in a human.

In one embodiment, the invention provides a use of lopinavir, or a pharmaceutically acceptable salt thereof, in combination with a compound that inhibits cytochrome P-450 (e.g. ritonavir) or a pharmaceutically acceptable salt thereof, to prepare a medicament for a human useful for reducing a dose between about 40 to 60% of 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof, following administration to the human.

In one embodiment, the invention provides lopinavir, or a pharmaceutically acceptable salt thereof, in combination with a compound that inhibits cytochrome P-450 (e.g. ritonavir) or a pharmaceutically acceptable salt thereof, for use in reducing a dose between about 40 to 60% of 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof, following administration to a human.

In one embodiment, the invention provides the use of 6-(3-Chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof; lopinavir or a pharmaceutically acceptable salt thereof; and a compound that inhibits cytochrome P-450 for the prophylactic or therapeutic treatment of a viral infection in a human.

In one embodiment, the invention provides 6-(3-Chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof; lopinavir or a pharmaceutically acceptable salt thereof; and a compound that inhibits cytochrome P-450 for use in the prophylactic or therapeutic treatment of a viral infection in a human.

In one embodiment, the invention provides an anti-viral agent(s) comprising (a) 6-(3-Chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof, which is used in combination with (b) lopinavir or a pharmaceutically acceptable salt thereof and (c) a compound that inhibits cytochrome P-450 for use in the prophylactic or therapeutic treatment of a viral infection in a human.

In one embodiment, the invention provides a compound 6-(3-Chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof, which is used in combination with lopinavir or a pharmaceutically acceptable salt thereof and a compound that inhibits cytochrome P-450 for use in the prophylactic or therapeutic treatment of a viral infection in a human.

In one embodiment, the invention provides the use of lopinavir or a pharmaceutically acceptable salt thereof, in combination with a compound that inhibits cytochrome P-450 (e.g. ritonavir) or a pharmaceutically acceptable salt thereof, to prepare a medicament useful for improving the pharmacokinetics of 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof, following administration to a human.

### DETAILED DESCRIPTION OF THE INVENTION

As used herein, the term “co-administer” refers to administration of two or more agents within a 24 hour period of each other, for example, as part of a clinical treatment regimen. In other embodiments, “co-administer” refers to administration within 2 hours of each other. In other embodiments, “co-administer” refers to administration within 30 minutes of each other. In other embodiments, “co-administer” refers to administration within 15 minutes of each other. In other embodiments, “co-administer” refers to administration at the same time, either as part of a single formulation or as multiple formulations that are administered by the same or different routes.

The term “lopinavir” refers to (2S)—N-[(2S,4S,5S)-5-{[2-(2,6-dimethyl-phenoxy)acetyl]amino}-4-hydroxy-1,6-diphenyl-hexan-2-yl]-3-methyl-2-(2-oxo-1,3-diazinan-1-yl)butanamide.

The term “ritonavir” refers to 1,3-thiazol-5-ylmethyl[3-hydroxy-5-[3-methyl-2-[methyl-[(2-propan-2-yl-1,3-thiazol-4-yl)methyl]carbamoyl]amino-butanoyl]amino-1,6-diphenyl-hexan-2-yl]aminoformate.

The term “unit dosage form” refers to a physically discrete unit, such as a capsule, tablet, or solution that is suitable as a unitary dosage for a human patient, each unit containing a predetermined quantity of one or more active ingredient(s) calculated to produce a therapeutic effect, in association with at least one pharmaceutically acceptable diluent or carrier, or combination thereof.

If desired, the effective daily dose of the Compound may be administered as two, three, four, five, six, or more sub-doses administered separately at appropriate intervals throughout the day, optionally, in unit dosage forms.

The concentration of the Compound in the bloodstream may be measured as the plasma concentration (ng/mL). Pharmacokinetic parameters for determining the plasma concentration include, but are not limited to, the maximum observed plasma concentration (Cₘₐₓ), observed plasma concentration at the end of the dosing interval or “trough” concentration (Cₜₐᵤ or Cₘᵢₙ), area under the plasma concentration time curve (AUC) from time zero up to the last quantifiable time point (AUC₀₋ₗₐₛₜ), AUC from time zero to infinity (AUC_{0-inf}), time of maximum observed plasma concentration after administration (tₘₐₓ), and half-life of the Compound in plasma (t_{1/2}).

Administration of the Compound with food according to the methods of the invention may also increase absorption of the Compound. Absorption of the Compound may be measured by the concentration attained in the bloodstream over time after administration of the Compound. An increase in absorption by administration of the Compound with food may also be evidenced by an increase in Cₘₐₓ and/or AUC_{0-inf} of the Compound as compared to the values if the Compound was administered without food. Typically protease inhibitors are administered with food.

Compounds that Inhibit Cytochrome P-450

As used herein, “Compounds that inhibit cytochrome P-450” include compounds that decrease the metabolism of Compound 1 by cytochrome P450, in particular, the metabolism of Compound 1 by cytochrome P450 3A. Accordingly, the term includes inhibitors of cytochrome P450, as well as substrates for cytochrome P450 and other compounds that decrease the metabolism of Compound 1 by cytochrome P450. A number of such compounds are known: see for example http://medicine.iupui.edu/flockhart/table.htm; and International Patent Application Publication Number WO 2008/010921.

Representative compounds include, cimetidine, fluoroquinolones, fluvoxamine, ticlopidine, thiotepa, ticlopidine, gemfibrozil, montelukast, fluoxetine, fluvoxamine, ketoconazole, lansoprazole, omeprazole, ticlopidine, amiodarone, fluconazole, isoniazid, amiodarone, buproprion, chlorpheniramine, cimetidine, clomipramine, duloxetine, fluoxetine, haloperidol, methadone, mibefradil, paroxetine, quinidine, ritonavir, disulfuram, indinavir, nelfinavir, amiodarone, cimetidine, clarithromycin, diltiazem, erythromycin, fluvoxamine, itraconazole, ketoconazole, mibefradil, nefazodone, troleandomycin, and verapamil.

A specific sub-set of cytochrome P-450 inhibitors that are useful in the methods of the invention includes ketoconazole, itraconazole, clarithromycin, telithromycin, indinavir, nelfinavir, saquinavir, nefazadone, erythromycin and ritonavir, and pharmaceutically acceptable salts thereof.

Another specific sub-set of cytochrome P-450 inhibitors that are useful in the methods of the invention includes the HIV protease inhibitors indinavir, nelfinavir, saquinavir, and ritonavir.

One specific agent that blocks Cytochrome P-450 activity and that is useful in the methods of the invention is ritonavir, or a pharmaceutically acceptable salt thereof. A specific dose of ritonavir that can be used according to the invention is 100±50 mg of ritonavir or a pharmaceutically acceptable salt thereof. A specific dose of ritonavir that can be used according to the invention is 100±25 mg of ritonavir or a pharmaceutically acceptable salt thereof. A specific dose of ritonavir that can be used according to the invention is 100±10 mg of ritonavir or a pharmaceutically acceptable salt thereof.

Other specific agents that block Cytochrome P-450 activity and that are useful in the methods of the invention are reported in International Patent Application Publication Number WO 2008/010921. In one specific embodiment of the invention, the compound that inhibits cytochrome P-450 is a compound of the following formula:

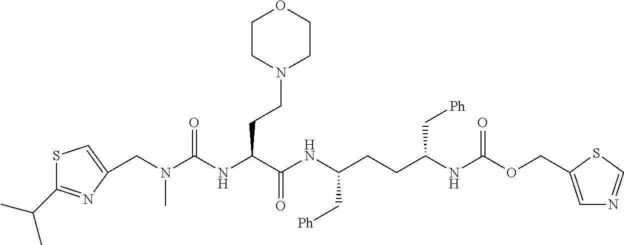

or a pharmaceutically acceptable salt thereof.

### Methods

In one embodiment the present invention provides a method for the treatment or prophylaxis of diseases, disorders, and conditions. An example of a disease, disorder, or condition includes, but is not limited to, a retrovirus infection, or a disease, disorder, or condition associated with a retrovirus infection. Retroviruses are RNA viruses and are generally classified into the alpharetrovirus, betaretrovirus, deltaretrovirus, epsilonretrovirus, gammaretrovirus, lentivirus, and spumavirus families. Examples of retroviruses include, but are not limited to, human immunodeficiency virus (HIV), human T-lymphotropic virus (HTLV), rous sarcoma virus (RSV), and the avian leukosis virus. In general, three genes of the retrovirus genome code for the proteins of the mature virus: gag (group-specific antigen) gene, which codes for the core and structural proteins of the virus; pol (polymerase) gene, which codes for the enzymes of the virus, including reverse transcriptase, protease, and integrase; and env (envelope) gene, which codes for the retrovirus surface proteins.

Retroviruses attach to and invade a host cell by releasing a complex of RNA and the pol products, among other things, into the host cell. The reverse transcriptase then produces double stranded DNA from the viral RNA. The double stranded DNA is imported into the nucleus of the host cell and integrated into the host cell genome by the viral integrase. A nascent virus from the integrated DNA is formed when the integrated viral DNA is converted into mRNA by the host cell polymerase and the proteins necessary for virus formation are produced by the action of the virus protease. The virus particle undergoes budding and is released from the host cell to form a mature virus.

In one embodiment, the invention comprises administering about 85 mg (e.g. ±10 mg, 5 mg, or 2 mg) of the Compound.

In one embodiment, the invention comprises administering about 175 mg (e.g. ±25 mg or 10 mg) of the Compound.

In one embodiment, the invention comprises administering about 170 mg (e.g. ±25 mg or 10 mg) of the Compound.

In one embodiment, the invention comprises administering about 400 mg (e.g. ±150 mg, 100 mg, 50 mg, or 10 mg) of lopinavir, or a pharmaceutically acceptable salt thereof.

In one embodiment, the invention comprises administering about 800 mg (e.g. ±150 mg, 100 mg, 50 mg, or 10 mg) of lopinavir, or a pharmaceutically acceptable salt thereof.

### Compositions

The active agents may be administered to a human in any conventional manner. While it is possible for the active agents to be administered as raw compounds, they are preferably administered as a pharmaceutical composition. A “pharmaceutical composition comprising the Compound” refers to a pharmaceutical composition comprising the Compound, or a pharmaceutically acceptable salt thereof, with one or more pharmaceutically acceptable carriers or diluents and optionally other therapeutic agents and/or components. The salt, carrier, or diluent should be acceptable in the sense of being compatible with the other ingredients and not deleterious to the recipient thereof. Examples of carriers or diluents for oral administration include cornstarch, lactose, magnesium stearate, talc, microcrystalline cellulose, stearic acid, povidone, crospovidone, dibasic calcium phosphate, sodium starch glycolate, hydroxypropyl cellulose (e.g., low substituted hydroxypropyl cellulose), hydroxypropylmethyl cellulose (e.g., hydroxypropylmethyl cellulose 2910), and sodium lauryl sulfate.

The pharmaceutical compositions may be prepared by any suitable method, such as those methods well known in the art of pharmacy, for example, methods such as those described in Gennaro et al., Remington's Pharmaceutical Sciences (18th ed., Mack Publishing Co., 1990), especially Part 8: Pharmaceutical Preparations and their Manufacture. Such methods include the step of bringing into association the Compound with the carrier or diluent and optionally one or more accessory ingredients. Such accessory ingredients include those conventional in the art, such as, fillers, binders, excipients disintegrants, lubricants, colorants, flavoring agents, sweeteners, preservatives (e.g., antimicrobial preservatives), suspending agents, thickening agents, emulsifying agents, and/or wetting agents.

The pharmaceutical compositions may provide controlled, slow release, or sustained release of the agents (e.g. the Compound) over a period of time. The controlled, slow release, or sustained release of the agents (e.g. the Compound) may maintain the agents in the bloodstream of the human for a longer period of time than with conventional formulations. Pharmaceutical compositions include, but are not limited to, coated tablets, pellets, solutions, powders, and capsules, and dispersions of the Compound in a medium that is insoluble in physiologic fluids or where the release of the therapeutic compound follows degradation of the pharmaceutical composition due to mechanical, chemical, or enzymatic activity.

The pharmaceutical composition of the invention may be, for example, in the form of a pill, capsule, solution, powder, or tablet, each containing a predetermined amount of the Compound. In an embodiment of the invention, the pharmaceutical composition is in the form of a tablet comprising the Compound and the components of the tablet utilized and described in the Examples herein.

For oral administration, fine powders or granules may contain diluting, dispersing, and or surface active agents and may be present, for example, in water or in a syrup, in capsules or sachets in the dry state, or in a nonaqueous solution or suspension wherein suspending agents may be included, or in tablets wherein binders and lubricants may be included.

When administered in the form of a liquid solution or suspension, the formulation may contain the Compound and purified water. Optional components in the liquid solution or suspension include suitable sweeteners, flavoring agents, preservatives (e.g., antimicrobial preservatives), buffering agents, solvents, and mixtures thereof. A component of the formulation may serve more than one function. For example, a suitable buffering agent also may act as a flavoring agent as well as a sweetener.

Suitable sweeteners include, for example, saccharin sodium, sucrose, and mannitol. A mixture of two or more sweeteners may be used. The sweetener or mixtures thereof are typically present in an amount of from about 0.001% to about 70% by weight of the total composition. Suitable flavoring agents may be present in the pharmaceutical composition to provide a cherry flavor, cotton candy flavor, or other suitable flavor to make the pharmaceutical composition easier for a human to ingest. The flavoring agent or mixtures thereof are typically present in an amount of about 0.0001% to about 5% by weight of the total composition.

Suitable preservatives include, for example, methylparaben, propylparaben, sodium benzoate, and benzalkoniyum chloride. A mixture of two or more preservatives may be used. The preservative or mixtures thereof are typically present in an amount of about 0.0001% to about 2% by weight of the total composition.

Suitable buffering agents include, for example, citric acid, sodium citrate, phosphoric acid, potassium phosphate, and various other acids and salts. A mixture of two or more buffering agents may be used. The buffering agent or mixtures thereof are typically present in an amount of about 0.001% to about 4% by weight of the total composition.

Suitable solvents for a liquid solution or suspension include, for example, sorbitol, glycerin, propylene glycol, and water. A mixture of two or more solvents may be used. The solvent or solvent system is typically present in an amount of about 1% to about 90% by weight of the total composition.

The pharmaceutical composition may be co-administered with adjuvants. For example, nonionic surfactants such as polyoxyethylene oleyl ether and n-hexadecyl polyethylene ether may be administered with or incorporated into the pharmaceutical composition to artificially increase the permeability of the intestinal walls. Enzymatic inhibitors may also be administered with or incorporated into the pharmaceutical composition.

In one embodiment the invention provides a pharmaceutical composition comprising 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof; lopinavir or a pharmaceutically acceptable salt thereof; and a pharmaceutically acceptable carrier or diluent.

In one embodiment of the invention the pharmaceutical composition comprises 85±10 mg of 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof.

In one embodiment of the invention the pharmaceutical composition comprises 85±5 mg of 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof.

In one embodiment of the invention the pharmaceutical composition comprises 85±2 mg of 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof.

In one embodiment of the invention the pharmaceutical composition comprises 175±25 mg of 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof.

In one embodiment of the invention the pharmaceutical composition comprises 175±10 mg of 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof.

In one embodiment of the invention the pharmaceutical composition comprises 400±150 mg of 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof.

In one embodiment of the invention the pharmaceutical composition comprises 400±100 mg of 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof.

In one embodiment of the invention the pharmaceutical composition comprises 400±50 mg of 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof.

In one embodiment of the invention the pharmaceutical composition comprises 400±10 g of 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof.

In one embodiment of the invention the pharmaceutical composition comprises 800±50 mg of 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof.

In one embodiment of the invention the pharmaceutical composition comprises 800±20 mg of 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof.

In one embodiment of the invention the pharmaceutical composition comprises 100±50 mg of ritonavir or a pharmaceutically acceptable salt thereof.

In one embodiment of the invention the pharmaceutical composition comprises 100±25 mg of ritonavir or a pharmaceutically acceptable salt thereof.

In one embodiment of the invention the pharmaceutical composition comprises 100±10 mg of ritonavir or a pharmaceutically acceptable salt thereof.

### Kits

In one embodiment the invention provides a kit comprising: (1) 6-(3-chloro-2-fluorobenzyl)-1-[(2S)-1-hydroxy-3-methylbutan-2-yl]-7-methoxy-4-oxo-1,4-dihydroquinoline-3-carboxylic acid, or a pharmaceutically acceptable salt thereof; (2) lopinavir, or a pharmaceutically acceptable salt thereof; (3) one or more containers; and (4) prescribing information regarding administering the 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof with the lopinavir or a pharmaceutically acceptable salt thereof.

In one embodiment, the kit comprises 85±10 mg of 6-(3-chloro-2-fluorobenzyl)-1-[(2S)-1-hydroxy-3-methylbutan-2-yl]-7-methoxy-4-oxo-1,4-dihydroquinoline-3-carboxylic acid, or a pharmaceutically acceptable salt thereof.

In one embodiment, the kit comprises 85±5 mg of 6-(3-chloro-2-fluorobenzyl)-1-[(2S)-1-hydroxy-3-methylbutan-2-yl]-7-methoxy-4-oxo-1,4-dihydroquinoline-3-carboxylic acid, or a pharmaceutically acceptable salt thereof.

In one embodiment, the kit comprises 85±2 mg of 6-(3-chloro-2-fluorobenzyl)-1-[(2S)-1-hydroxy-3-methylbutan-2-yl]-7-methoxy-4-oxo-1,4-dihydroquinoline-3-carboxylic acid, or a pharmaceutically acceptable salt thereof.

In one embodiment, the kit comprises 175±25 mg of 6-(3-chloro-2-fluorobenzyl)-1-[(2S)-1-hydroxy-3-methylbutan-2-yl]-7-methoxy-4-oxo-1,4-dihydroquinoline-3-carboxylic acid, or a pharmaceutically acceptable salt thereof.

In one embodiment, the kit comprises 175±10 mg of 6-(3-chloro-2-fluorobenzyl)-1-[(2S)-1-hydroxy-3-methylbutan-2-yl]-7-methoxy-4-oxo-1,4-dihydroquinoline-3-carboxylic acid, or a pharmaceutically acceptable salt thereof.

In one embodiment, the kit comprises 400±150 mg of lopinavir or a pharmaceutically acceptable salt thereof.

In one embodiment, the kit comprises 400±100 mg of lopinavir or a pharmaceutically acceptable salt thereof.

In one embodiment, the kit comprises 400±50 mg of lopinavir or a pharmaceutically acceptable salt thereof.

In one embodiment, the kit comprises 400±10 mg of lopinavir or a pharmaceutically acceptable salt thereof.

In one embodiment, the kit comprises 800±50 mg of lopinavir or a pharmaceutically acceptable salt thereof.

In one embodiment, the kit comprises 800±20 mg of lopinavir or a pharmaceutically acceptable salt thereof.

In one embodiment, the kit further comprises a compound that inhibits cytochrome P-450 (e.g. ritonavir) or a pharmaceutically acceptable salt thereof.

In one embodiment, the kit comprises 100±50 mg of ritonavir or a pharmaceutically acceptable salt thereof.

In one embodiment, the kit comprises 100±25 mg of ritonavir or a pharmaceutically acceptable salt thereof.

In one embodiment, the kit comprises 100±10 mg of ritonavir or a pharmaceutically acceptable salt thereof.

In one embodiment the invention provides a kit comprising: (1) a unit dosage form comprising 6-(3-chloro-2-fluorobenzyl)-1-[(2S)-1-hydroxy-3-methylbutan-2-yl]-7-methoxy-4-oxo-1,4-dihydroquinoline-3-carboxylic acid, or a pharmaceutically acceptable salt thereof; (2) lopinavir, or a pharmaceutically acceptable salt thereof; (3) one or more containers; and (4) prescribing information regarding administering the 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof with the lopinavir or a pharmaceutically acceptable salt thereof.

In one embodiment, the unit dosage form comprises 85±10 mg of 6-(3-chloro-2-fluorobenzyl)-1-[(2S)-1-hydroxy-3-methylbutan-2-yl]-7-methoxy-4-oxo-1,4-dihydroquinoline-3-carboxylic acid, or a pharmaceutically acceptable salt thereof.

In one embodiment, the unit dosage form comprises 85±5 mg of 6-(3-chloro-2-fluorobenzyl)-1-[(2S)-1-hydroxy-3-methylbutan-2-yl]-7-methoxy-4-oxo-1,4-dihydroquinoline-3-carboxylic acid, or a pharmaceutically acceptable salt thereof.

In one embodiment, the unit dosage form comprises 85±2 mg of 6-(3-chloro-2-fluorobenzyl)-1-[(2S)-1-hydroxy-3-methylbutan-2-yl]-7-methoxy-4-oxo-1,4-dihydroquinoline-3-carboxylic acid, or a pharmaceutically acceptable salt thereof.

In one embodiment, the unit dosage form comprises 175±25 mg of 6-(3-chloro-2-fluorobenzyl)-1-[(2S)-1-hydroxy-3-methylbutan-2-yl]-7-methoxy-4-oxo-1,4-dihydroquinoline-3-carboxylic acid, or a pharmaceutically acceptable salt thereof.

In one embodiment, the unit dosage form comprises 175±10 mg of 6-(3-chloro-2-fluorobenzyl)-1-[(2S)-1-hydroxy-3-methylbutan-2-yl]-7-methoxy-4-oxo-1,4-dihydroquinoline-3-carboxylic acid, or a pharmaceutically acceptable salt thereof.

In one embodiment, the kit comprises a unit dosage form that comprises 400±150 mg of lopinavir or a pharmaceutically acceptable salt thereof.

In one embodiment, the kit comprises a unit dosage form that comprises 400±100 mg of lopinavir or a pharmaceutically acceptable salt thereof.

In one embodiment, the kit comprises a unit dosage form that comprises 400±50 mg of lopinavir or a pharmaceutically acceptable salt thereof.

In one embodiment, the kit comprises a unit dosage form that comprises 400±10 mg of lopinavir or a pharmaceutically acceptable salt thereof.

In one embodiment, the kit comprises a unit dosage form that comprises 800±50 mg of lopinavir or a pharmaceutically acceptable salt thereof.

In one embodiment, the kit comprises a unit dosage form that comprises 800±20 mg of lopinavir or a pharmaceutically acceptable salt thereof.

In one embodiment, the kit further comprises a unit dosage form that comprises a compound that inhibits cytochrome P-450 (e.g. ritonavir) or a pharmaceutically acceptable salt thereof.

In one embodiment, the kit comprises a unit dosage form that comprises 100±50 mg of ritonavir or a pharmaceutically acceptable salt thereof.

In one embodiment, the kit comprises a unit dosage form that comprises 100±25 mg of ritonavir or a pharmaceutically acceptable salt thereof.

In one embodiment, the kit comprises a unit dosage form that comprises 100±10 mg of ritonavir or a pharmaceutically acceptable salt thereof.

The invention will now be illustrated by the following non-limiting examples.

### Example 1

### A Pharmacokinetic Interaction between Lopinavir/r and the Compound

The effects of coadministration of lopinavir/r (LPV/r) with the Compound were determined. This study evaluated the safety and steady-state pharmacokinetics of the coadministered Compound and LPV/r.

### Methods

Within two groups, healthy volunteers were randomized to follow one of two consecutive 14-day treatment periods: the Compound/r (125/100 mg QD) and the Compound (125 mg QD)+LPV/r (400/100 mg BID) in group 1 or LPV/r (400/100 mg BID) and the Compound (125 mg QD)+LPV/r (400/100 mg BID) in group 2. Lack of PK alteration bounds for 90% confidence intervals (CI) about the geometric mean ratio (GMR) (coadministration: alone) were 70-143% for the Compound and 80-125% for LPV.

### Results

Twenty-seven of 32 enrolled subjects completed the study. The most frequent treatment-related adverse events were GI disorders (˜62% in LPV/r±the Compound) and headaches (˜44% in the Compound/r treatment). Pharmacokinetic results were as follows:

% GMR (90% CI)

| | the Compound | | LPV | | RTV | |
|---|---|---|---|---|---|---|
| | (n = 14) | | (n = 13) | | (n = 13) | |
| AUCₜₐᵤ | 175 | (150, 204) | 96.6 | (85.3, 109) | 103 | (87.0, 121) |
| Cₘₐₓ | 152 | (129, 179) | 99.2 | (88, 112) | 114 | (86.9, 149) |
| Cₜₐᵤ | 238 | (181, 313) | 92.3 | (78.7, 108) | 88.3 | (74.4, 105) |

The Compound exposures were substantially elevated upon coadministration with LPV/r, possibly via LPV-mediated inhibition of UGT1A1/3 metabolism as the Compound undergoes biotransformation through glucuronidation as well as oxidative metabolism.

A reduced dose of the Compound was selected through modeling a variety of doses using compartmental modeling in WinNonlin (Pharsight Corporation, Mountain View, Calif., USA) incorporating the observed drug-drug interaction data with lopinavir from the above results. Consideration was given to achieving equivalent Compound exposures in patients receiving and not receiving lopinavir using pharmacokinetic (bio-) equivalence comparisons (Pharsight Corporation, Mountain View, Calif., USA). Consideration was also given to minimizing the number of individuals with extreme outliers in (low or high) exposures. Thus, the 85 mg and 150 mg doses of the Compound with lopinavir/r are expected to provide similar systemic exposures (AUC) to the 150 mg and 300 mg ritonavir-boosted doses without lopinavir. LPV and RTV exposures were unaltered when coadministered with the Compound; LPV trough concentrations were maintained above recommended target troughs. Accordingly, a reduction of about 40-60% in the dose of the Compound can be administered with lopinavir while maintaining an equivalent exposure.

### Conclusion

A reduced dose of the Compound (e.g. 85±10 mg) can be administered to achieve a comparable systemic exposure when the Compound is administered with lopinavir. It is believed that lopinavir improves the pharmacokinetic exposure of the Compound by blocking the UGT1A1/3 metabolic pathway of the compound.

Similar studies were carried out to determine the effect of five different protease inhibitors on the pharmacokinetics of the Compound. These studies employed various doses of ritonavir (100 mg QD to 200 mg BID). Of the five protease inhibitors that were tested, three were found to have no effect on the pharmacokinetics of the Compound. Only two (including lopinavir) of the five were found to have an improved pharmacokinetic effect on the Compound.

### Example 2

### Representative Example of the Formulation of 6-(3-chloro-2-fluorobenzyl)-1-[(2S)-1-hydroxy-3-methylbutan-2-yl]-7-methoxy-4-oxo-1,4-dihydroquinoline-3-carboxylic acid

TABLE 1

| Component | Function | Amount Per Tablet | |
|---|---|---|---|
| The Compound | Drug substance | 125.0 | mg |
| Mannitol USP | Diluent | 67.4 | mg |
| Colloidal Silicon Dioxide NF | Glidant | 15.7 | mg |
| Sodium lauryl sulfate NF | Surfactant | 6.1 | mg |
| Crospovidone NF | Disintegrant | 15.6 | mg |
| Hypromellose 2910 USP | Binder | 12.6 | mg |
| Purified water*¹ USP | Binder agent | — | |
| Croscarmellose sodium NF | Disintegrant | 61.7 | mg |
| Magnesium Stearate NF | Lubricant | 2.3 | mg |
| Total tablet weight | | 306.4 | mg |

*¹The purified water is removed during processing.

The Compound was first micronized with a jet mill. The micronized compound was mixed with Mannitol, Crospovidone, and Colloidal Silicon Dioxide in a polyethylene (PE) bag and then passed though a 500 μm screen three times. Hypromellose 2910 was separately dissolved in purified water by stirring and sodium lauryl sulfate was added and dissolved. The Mannitol/Crospovidone/Colloidal Silicon Dioxide/the Compound mixture was placed in a fluidized-bed granulator and was granulated using the Hypromellose/sodium lauryl sulfate solution. After granulation, the wet granulates were dried in the same granulator. The dried granules were passed through a 500 μm screen.

The screened granules were then mixed with croscarmellos sodium in a blender and magnesium stearate was added to the blender and mixed. The granules were compressed into tablets using a rotary tableting machine.

All references, including publications, patent applications, and patents, cited herein are hereby incorporated by reference in their entirety.

The use of the terms “a” and “an” and “the” and similar referents in the context of describing the invention (including the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein may be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The embodiments within the specification provide an illustration of embodiments of the invention and should not be construed to limit the scope of the invention. The skilled artisan recognizes that many other embodiments are encompassed by the claimed invention and that it is intended that the specification and examples be considered as exemplary only, with the true scope and spirit of the invention being indicated by the following claims.

1-21. (canceled)

22. A pharmaceutical composition comprising 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof; lopinavir or a pharmaceutically acceptable salt thereof; and a pharmaceutically acceptable carrier or diluent.

23. The composition of claim 22 that comprises 85±10 mg of 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof.

24. The composition of claim 22 that comprises 175±25 mg of 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof.

25. The composition of claim 22 which comprises 400±150 mg of lopinavir or a pharmaceutically acceptable salt thereof.

26. The composition of claim 22 which comprises 800±50 mg of lopinavir or a pharmaceutically acceptable salt thereof.

27. The composition of claim 22 which further comprises a compound that inhibits cytochrome P-450.

28. The composition of claim 27 wherein the compound that inhibits cytochrome P-450 is ritonavir.

29. The composition of claim 28 which comprises 100±50 mg of ritonavir or a pharmaceutically acceptable salt thereof.

30. The composition of claim 27 wherein the compound that inhibits cytochrome P-450 is a compound of the following formula: 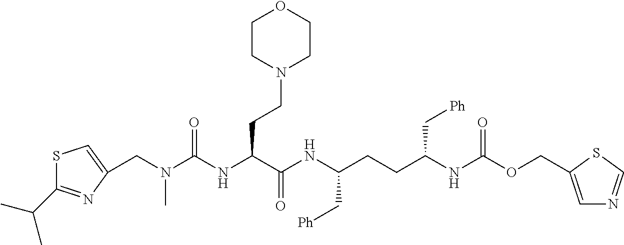
or a pharmaceutically acceptable salt thereof.

31-51. (canceled)

52. A kit comprising: (1) 6-(3-chloro-2-fluorobenzyl)-1-[(2S)-1-hydroxy-3-methylbutan-2-yl]-7-methoxy-4-oxo-1,4-dihydroquinoline-3-carboxylic acid, or a pharmaceutically acceptable salt thereof; (2) lopinavir, or a pharmaceutically acceptable salt thereof; (3) one or more containers; and (4) prescribing information regarding administering the 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof with the lopinavir or a pharmaceutically acceptable salt thereof.

53. The kit of claim 52 which comprises 85±10 mg of 6-(3-chloro-2-fluorobenzyl)-1-[(2S)-1-hydroxy-3-methylbutan-2-yl]-7-methoxy-4-oxo-1,4-dihydroquinoline-3-carboxylic acid, or a pharmaceutically acceptable salt thereof.

54. The kit of claim 52 which comprises 175±25 mg of 6-(3-chloro-2-fluorobenzyl)-1-[(2S)-1-hydroxy-3-methylbutan-2-yl]-7-methoxy-4-oxo-1,4-dihydroquinoline-3-carboxylic acid, or a pharmaceutically acceptable salt thereof.

55. The kit of claim 52 which comprises 400±150 mg of lopinavir or a pharmaceutically acceptable salt thereof.

56. The kit of claim 52 which comprises 800±50 mg of lopinavir or a pharmaceutically acceptable salt thereof.

57. The kit of claim 52 which further comprises a compound that inhibits cytochrome P-450.

58. The kit of claim 52 which further comprises ritonavir or a pharmaceutically acceptable salt thereof.

59. The kit of claim 58 which comprises 100±50 mg of ritonavir or a pharmaceutically acceptable salt thereof.

60. The kit of claim 57 wherein the compound that inhibits cytochrome P-450 is a compound of the following formula: 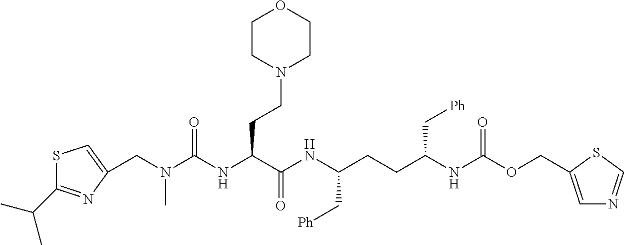
or a pharmaceutically acceptable salt thereof.

61. (canceled)

62. A method of treating a viral infection in a human comprising administering 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof, lopinavir, or a pharmaceutically acceptable salt thereof, and a compound that inhibits cytochrome P-450 to the human.

63-65. (canceled)

66. The method of claim 62 wherein 85±10 mg of 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof, are administered.

67. The method of claim 62 wherein 175±25 mg of 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof, are administered.

68. The method of claim 62 wherein 400±150 mg of lopinavir or a pharmaceutically acceptable salt thereof is administered.

69. The method of claim 62 wherein 800±50 mg of lopinavir or a pharmaceutically acceptable salt thereof is administered.

70. The method of claim 62 wherein the compound that inhibits cytochrome P-450 is ritonavir, or a pharmaceutically acceptable salt thereof.

71. The method of claim 70 wherein 100±50 mg of ritonavir or a pharmaceutically acceptable salt thereof is administered to the human.

72. The method of claim 62 wherein the 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof, and the lopinavir or a pharmaceutically acceptable salt thereof are coadministered.

73. The method of claim 72 wherein the 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof, and the lopinavir or a pharmaceutically acceptable salt thereof are administered within 15 minutes of each other.

74. The method of claim 62 wherein a single dosage form comprising the 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof, and the lopinavir or a pharmaceutically acceptable salt thereof is administered.

75. The method of claim 70 wherein the 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof, and the ritonavir or a pharmaceutically acceptable salt thereof are coadministered.

76. The method of claim 75 wherein the 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof, and the ritonavir or a pharmaceutically acceptable salt thereof are administered within 15 minutes of each other.

77. The method of claim 75 wherein a single dosage form comprising the 6-(3-chloro-2-fluorobenzyl)-1-[(2S)-1-hydroxy-3-methylbutan-2-yl]-7-methoxy-4-oxo-1,4-dihydroquinoline-3-carboxylic acid or a pharmaceutically acceptable salt thereof, and the ritonavir or a pharmaceutically acceptable salt thereof is administered.

78. The method of claim 70 wherein the lopinavir or a pharmaceutically acceptable salt thereof, and the ritonavir or a pharmaceutically acceptable salt thereof are coadministered.

79. The method of claim 78 wherein the lopinavir or a pharmaceutically acceptable salt thereof, and the ritonavir or a pharmaceutically acceptable salt thereof are administered within 15 minutes of each other.

80. The method of claim 78 wherein a single dosage form comprising the lopinavir or a pharmaceutically acceptable salt thereof, and the ritonavir or a pharmaceutically acceptable salt thereof is administered.

81. The method of claim 62 wherein the virus is human immunodeficiency virus (HIV).

82. The method of claim 62 wherein the compound that inhibits cytochrome P-450 is a compound of the following formula: 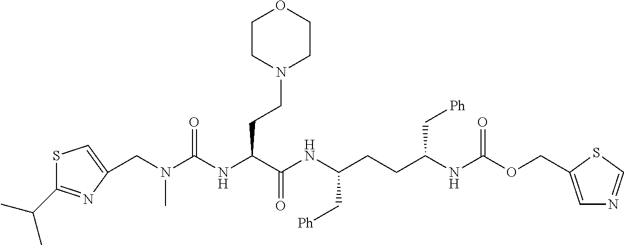
or a pharmaceutically acceptable salt thereof.
